(19) United States
(12) Patent Application Publication
(10) Pub. No.: US 2012/0195895 A1
(43) Pub. Date: Aug. 2, 2012

(54) Fusion Protein of an Anti-CD20 Antibody Fab Fragment and Lidamycin, a Method for Preparing the Same, and the Use Thereof

(76) Inventors: Chunzheng Yang, Tianjin (CN); Yongsu Zhen, Beijing (CN); Dongsheng Xiong, Tianjin (CN); Rongguang Shao, Beijing (CN); Zhenping Zhu, Tianjin (CN); Qingfang Miao, Beijing (CN); Xin Cheng, Tianjin (CN); Shenghua Zhang, Beijing (CN); Yuanfu Xu, Tianjin (CN); Hong Fang, Beijing (CN); Yingdai Gao, Tianjin (CN); Lianfang Jin, Beijing (CN)

(21) Appl. No.: 13/387,531

(22) PCT Filed: Jul. 27, 2010
(86) PCT No.: PCT/CN10/01141

§ 371 Date: Apr. 9, 2012

(30) Foreign Application Priority Data
Jul. 28, 2009 (CN) 200910157388.5

(51) Int. Cl. A61K 39/395; C12N 15/62; A61P 35/00; C12N 1/21; C12P 21/02; C07K 19/00; C12N 15/63

(52) U.S. Cl. 424/134.1; 530/387.3; 536/23.4; 435/320.1; 435/252.3; 435/252.33; 435/69.6

(57) ABSTRACT

The present invention relates to an anticancer drug, an energized fusion protein Anti-CD20(Fab)-LDM of lidamycin, a gene encoding the same; and further relates to a method for construction of the energized fusion protein in a genetic engineering manner and the use of the energized fusion protein. The applicant provides an anti-tumor drug with a good targeting ability by providing the energized fusion protein.

### TECHNICAL FIELD

The present invention relates to the field of oncology and biopharmacy. In particular, the present invention provides a fusion protein having an effect of targeting and killing tumors, a method for preparing the same, and the use thereof, and further provides a good candidate medicament for tumor-targeted therapy.

### BACKGROUND ART

Non-Hodgkin's Lymphoma (NHL) is a malignant tumor that starts in lymphoid tissue and whose morbidity and mortality rank fifth in malignant tumors. Conventional chemotherapy and radiotherapy are highly effective to NHL but have poor selectivity, because they might have some normal cells injured in vivo when killing tumor cells, and therefore generally lead to an obvious toxic side effect. Thus, tumor-targeted therapy becomes an important way to improve therapeutic effect.

In tumor-targeted therapy, the selection of a target for the therapy is crucial. It is known that most NHLs are originated from B lymphocyte and more than 95 percent of B cell NHL express CD20 antigen. Furthermore, CD20 is only expressed in pre-B lymphocyte, immature B lymphocyte, mature B lymphocyte and activated B lymphocyte but are not expressed in plasma cells, pluripotent stem cells or other tissues. Moreover, CD20 antigen is almost exposed to the surface and no free CD20 is present in human blood serum. Therefore, CD20 can be an effective target for treating B cell lymphoma.

Monoclonal antibodies associated with the tumor-related-antigens are most widely used in the tumor-targeted therapy. Since the anti-CD20 human-mouse chimeric antibody Rituximab was approved by US FDA in 1997, people highly desire for antibody drugs. In addition, it is a research hotspot to treat autoimmune diseases, such as rheumatoid arthritis, systemic lupus erythematosus (SLE) and so on, with anti-CD20 antibodies. This suggests that the therapeutic application of the anti-CD20 antibody will be further exploited. However, with the increase of the cases treated with Rituximab, the issue involving drug resistance is more obvious. To circumvent the drug resistance of Rituximab, new antibody drugs have been developed, for example, ¹³¹I labeled anti-CD20 Murine-antibody Bexxar and ⁹⁰Y labeled antibody Zevalin, which are different from Rituximab in mechanism. However, they also have weakness, such as high immunogenicity, one permitted injection only, serious toxic side effect, poor tolerance in patients. Therefore, it is urgent to develop a highly effective CD20-targeted medicament of a smaller size.

### SUMMARY OF THE INVENTION

Based on the situations described above, the applicant presents a strategy of combining a smaller Anti-CD20 antibody Fab fragment (targeting carrier) with a powerful antineoplastic agent (named “warhead” agent).

For the selection of a smaller antibody fragment, the applicant chose an anti-CD20 antibody fragment Fab as the targeting carrier. The Fab fragment is composed of heavy chain variable region (VH), constant region CH1, light chain variable region (VL), and constant region CL. The Fab fragment has advantages such as a small molecular weight, a strong penetrating capability, a short half-life in vivo, easy genetic modification in vitro and large-scale production by bacterial fermentation. Furthermore, due to small molecular weight, Fab is easy to penetrate the dense cancer cell barrier into the deep part of the solid tumor, and less HAMA reaction occurs due to low immunogenicity. Meanwhile, since the Fc fragment is absent, Fc-mediated receptor binding is avoided and therefore Fab can be quickly concentrated at the target site. Moreover, since Fab can be genetically modified easily, an active protein gene is linked to the gene encoding Fab by DNA recombination techniques and the resultant gene is expressed in a receptor to produce a targeted fusion protein. Therefore, it is promising to use an antibody Fab fragment as a tumor-targeted drug carrier.

As to the “warhead” agent, the applicant chose a highly potent “warhead” agent, lidamycin (LDM), which is also called C-1027 or C1027. LDM is an antibiotic of enediynes, which is produced by Streptomyces globisporus (accession number: CGMCC No. 0704) isolated from the soil in Qianjiang country in Hubei province in China. So far, LDM is the most potent anti-tumor antibiotic of macromolecular peptides for killing tumor cells ever reported. LDM consists of two parts: an active enediyne (AE) chromophone, which has cytotoxic effects but is unstable; and apoprotein (LDP) made up of 110 amino acid residues, which keeps chromophore stable. The chromophone and apoprotein are connected via non-covalent bonds, and the connection between them is specific and stable. The LDM can be dissociated into chromophone and apoprotein, both of which can also be reassembled into LDM. LDM is fit for the “warhead” agent due to its special molecular structure.

The applicant constructs a new fusion protein anti-CD20Fab-LDM by gene engineering techniques. Firstly, the CH1 fragment is obtained from the amplification of the recombinant plasmid pCANTAB 5E Fcd20 Fab′ containing anti-CD20 antibody Fab fragment and the LDP gene is obtained from the amplification of the plasmid pET30sngrldp (accession number: CGMCC No. 2010). Secondly, Fab-LDP gene is obtained by SOE-PCR, and then the fragment is reassembled into a plasmid pCANTAB 5E Fcd20 Fab′ in which CH1 gene is removed, thereby obtaining the plasmid pCANTAB 5E-Fab-LDP containing anti-CD20(Fab)-LDP. Furthermore, the resultant plasmid is transduced into the expression host bacterial strain, and a soluble Fab-LDP fusion protein expressed is obtained by changing the cultivation temperature, medium component and incubation time to optimize the culture condition. Finally, the fusion protein is reassembled with AE molecule to produce the energized fusion protein Fab-LDM. In animal tests, the energized fusion protein Fab-LDM according to the invention retains the targeting activity of an anti-CD20 antibody and cytotoxic activity of LDM. As compared to anti-CD20Fab or LDM at the same dose, the Fab-LDM according to the invention shows stronger tumor-suppressive effect. Therefore, the present invention provides a new drug candidate for tumor-targeted therapy, derived from the fusion of anti-CD20 antibody Fab fragment and LDM, which can be used to treat tumors.

### DESCRIPTION OF THE DRAWINGS

FIG. 1 is the restriction enzyme analysis result for the recombinant expression plasmid pCANTAB 5E-Fab-LDP, wherein

Lane 1 represents the DNA molecular weight marker;

Lane 2 represents the recombinant plasmid pCANTAB 5E-Fab-LDP /apal+sphl.

FIG. 2a is the SDS-PAGE analysis result for the expression product of the fusion protein Fab-LDP, wherein

Lane 1: the periplasmic proteins of the recombinant strain pCANTAB 5E-Fab-LDP after purification under a non-reductive condition;

Lane 2: the non-reductive effluent of the periplasmic proteins of recombinant strain pCANTAB 5E-Fab-LDP;

Lane 3: the non-reductive periplasmic proteins of the recombinant strain pCANTAB 5E-Fab-LDP before loading;

Lane 4: the periplasmic proteins of the recombinant strain pCANTAB 5E-Fab-LDP after reduction and purification;

Lane 5: the reductive effluent of the periplasmic proteins of the recombinant strain pCANTAB 5E-Fab-LDP;

Lane 6: the reductive periplasmic proteins of the recombinant strain pCANTAB 5E-Fab-LDP before loading.

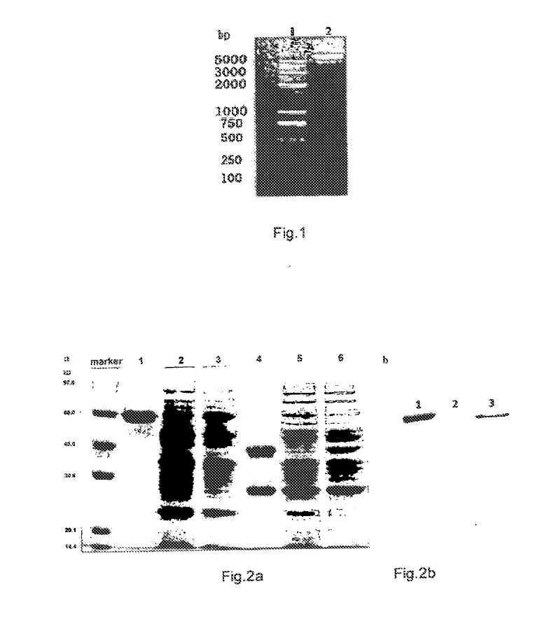

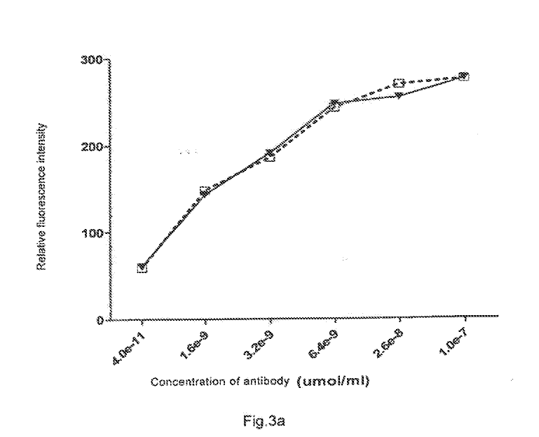

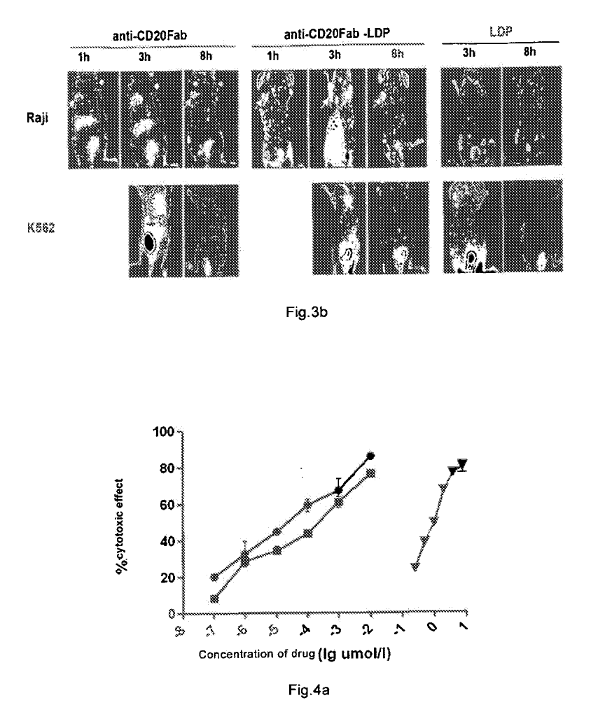

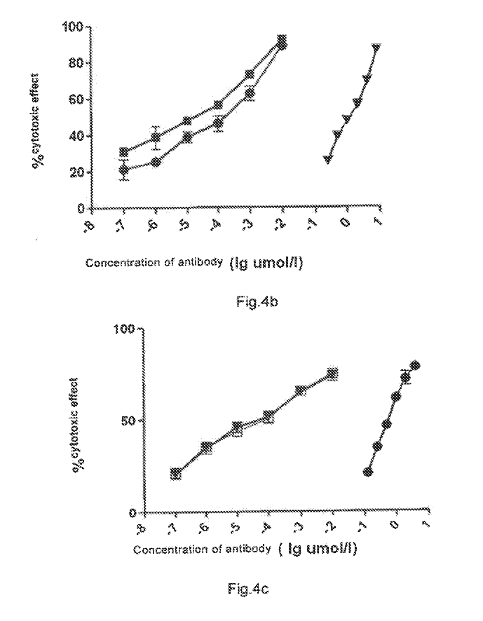

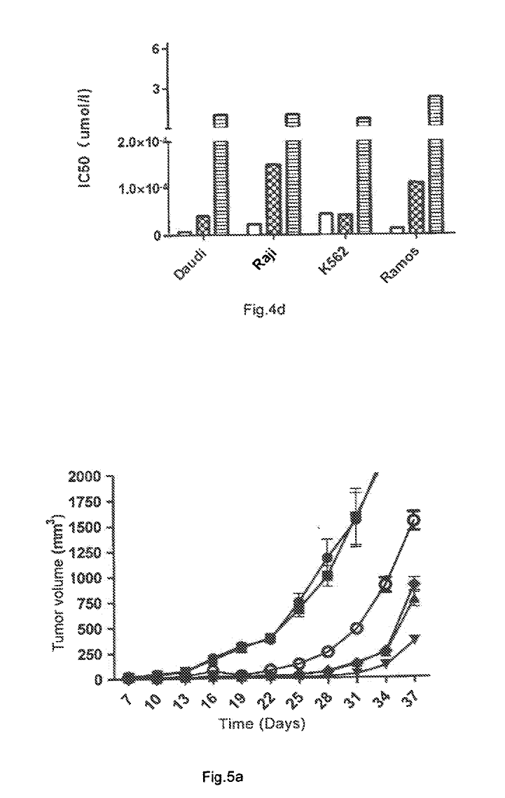

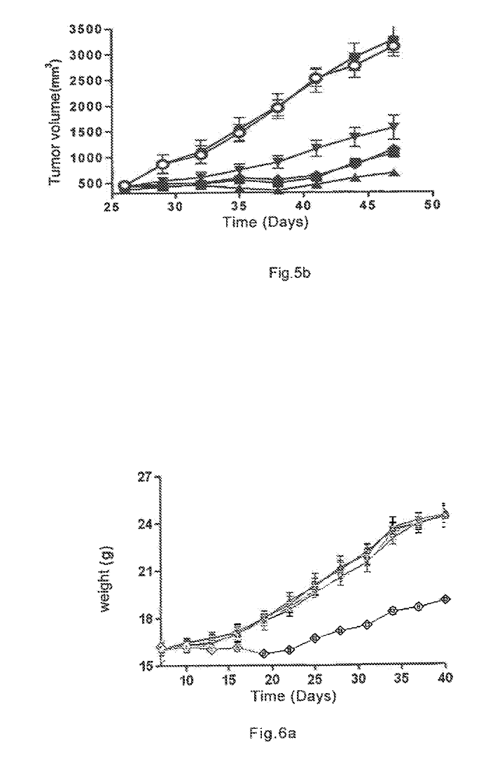

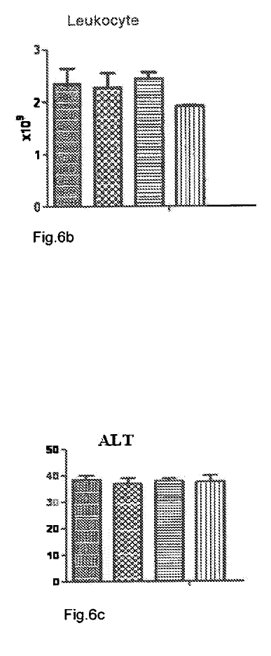

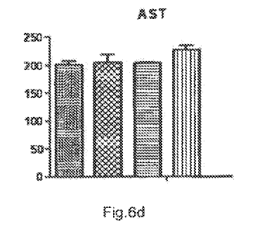

FIG. 2b is the Western blot analysis result of the expression product of the fusion protein Fab-LDP, wherein

Lane 1: the periplasmic proteins of the recombinant strain pCANTAB 5E-Fab-LDP after purification under a non-reductive condition;

Lane 2: the non-reductive effluent of the periplasmic proteins of the recombinant strain pCANTAB 5E-Fab-LDP;

Lane 3: the non-reductive periplasmic proteins of recombinant strain pCANTAB 5E-Fab-LDP before loading.

FIG. 3a is the FACS analysis result of the activity of anti-CD20Fab and Fab-LDP to bind Raji cells, respectively, wherein

▾ represents Fab-LDP; □ represents anti-CD20Fab.

FIG. 3b is the result of dynamic distribution of anti-CD20Fab, anti-CD20Fab-LDP and LDP in the body of Tumor-bearing Nude Mice, as observed in the in vivo imaging instrument.

FIG. 4a represents cytotoxic effect of the energized fusion protein Fab-LDM on Raji cells, wherein

[symbol] Fab-LDM

[symbol] LDM

[symbol] ADR

FIG. 4b represents cytotoxic effect of the energized fusion protein Fab-LDM on Daudi cells, wherein

[symbol] LDM

[symbol] Fab-LDM

[symbol] ADR

FIG. 4c represents cytotoxic effect of the energized fusion protein Fab-LDM on K562 cell, wherein

[symbol] Fab-LDM

[symbol] LDM

[symbol] ADR

FIG. 4d represents the comparative result of IC₅₀ of the energized fusion protein Fab-LDM and LDM in different cells, wherein

[symbol] Fab-LDM

[symbol] LDM

[symbol] ADR

FIG. 5a represents The therapeutic effect of the energized fusion protein Fab-LDM against CD20⁺ B-cell lymphoma xenografts in nude mice at early stage, wherein

represents PBS;

▪ represents anti-CD20Fab 4 nmol/kg;

▴ A represents Fab-LDM 2 nmol/kg;

♦ represents LDM 4 nmol/kg;

represents Fab-LDM 4 nmol/kg;

◯ represents LDM 2 nmol/kg.

FIG. 5b represents the therapeutic effect of the energized fusion protein Fab-LDM against CD20⁺ B-cell lymphoma xenograftsin nude mice at later stage, therein

represents PBS;

◯ represents anti-CD20Fab 4 nmol/kg;

▪ represents Fab-LDM 2 nmol/kg;

♦ represents LDM 4 nmol/kg;

▴ represents Fab-LDM 4 nmol/kg;

▾ represents LDM 2 nmol/kg.

FIG. 6a represents the effect of the energized fusion protein Fab-LDM on the body weight of the nude mice, wherein

[symbol] control

[symbol] Fab 4 nmol/kg

[symbol] Fab-LDM 2 nmol/kg

[symbol] Fab-LDM 4 nmol/kg

[symbol] LDM 2 nmol/kg

[symbol] LDM 4 nmol/kg

FIG. 6b represents the effect of the energized fusion protein Fab-LDM on leukocytes in the nude mice, wherein

[symbol] PBS

[symbol] Fab 4 nmol/kg

[symbol] Fab-LDM 2 nmol/kg

[symbol] Fab-LDM 4 nmol/kg

FIG. 6c represents the effect of the energized fusion protein Fab-LDM on alanine transaminase (ALT) of the nude mice, wherein

[symbol] PBS

[symbol] Fab 4 nmol/kg

[symbol] Fab-LDM 2 nmol/kg

[symbol] Fab-LDM 4 nmol/kg

FIG. 6d represents the effect of the energized fusion protein Fab-LDM on aspartate transaminase (AST), wherein

[symbol] PBS

[symbol] Fab 4 nmol/kg

[symbol] Fab-LDM 2 nmol/kg

[symbol] Fab-LDM 4 nmol/kg

### DETAILED DESCRIPTION OF THE INVENTION

The terms mentioned in the present invention should be understood in accordance with the following definitions.

In the present context, the term “LDM” is equivalent to “lidamycin”, “LDP-AE”, or “lidamycin apoprotein, the apoprotein of which is bound to chromophore AE”. The chromophone and apoprotein are connected via non-covalent bonds, and the connection between them is specific and stable. The LDM can be dissociated into chromophone and apoprotein, and both of which can also be reassembled into LDM.

The term “AE” mentioned in the present context refers to the chromophore as shown in formula (I).

The chemical name of LDM chromophore is (2R,7S,9R,10R)-7-amino-7,8-(2*-chloro-6*-hydroxyl-1*,4*-phenylene)-10-(4′-deoxy-4′-dimethylamino-5′,5′-dimethyl-ribopyranosyl)-4,8-oxa-5-oxo-1,11,13-triene-15,18-diyne-tricyclic[7,7,3,0^{10,14}]-2-nonadecyl alcohol-2″,3″-dihydrogen-7″-methoxy-2″-methylene-3″-oxo-1″,4″-benzoxazine-5″-carboxylate. The molecular formula: C₄₃H₄₂O₁₃N3Cl.

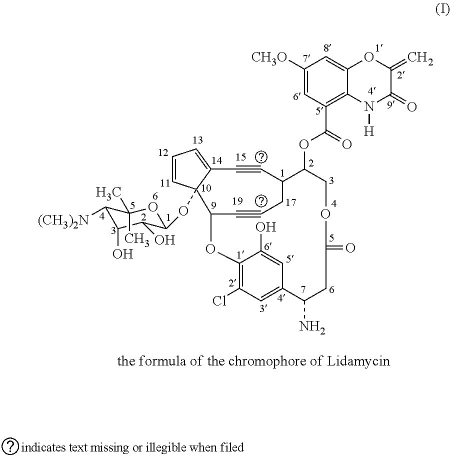

AE is produced by the wild type LDM-producing bacteria, which are natively bound onto lidamycin apoprotein, and the chromophore AE in a free state can be obtained by treating lidamycin with organic solvents such as cold methanol and so on under hypothermic condition. The free chromophore AE can be assembled with the lidamycin apoprotein LOP having AE removed or LDP produced by genetic engineering (which is fused with other protein fragments or not) into the same active form as the natural lidamycin under hypothermic condition. This reassembly is called “energized”.

In the present context, the term “Fab” is equivalent to “antiCD20(Fab)”, “antiCD20Fab”, or “anti-CD20 antibody Fab fragment”, which is anti-CD20 antibody Fab fragment of the amino acid residues from positions 24 to 467 of SEQ ID NO:1.

In the present context, the term “Fab-LDP” is equivalent to “antiCD20(Fab)-LDP”, “anti-CD20(Fab)-LDP”, “a fusion protein of anti-CD20 antibody Fab and lidamycin apoprotein”, which is a fusion protein as set forth in SEQ ID NO:1.

In the present context, the term “Fab-LDM” as used herein is equivalent to “energized fusion protein Fab-LDM”, “anti-CD20Fab-LDM”, “anti-CD20(Fab)-LDM”, “a fusion protein of anti-CD20 antibody Fab and lidamycin”, which is a fusion protein as set forth in SEQ ID NO:1, wherein the chromophore AE is bound to the lidamycin apoprotein.

1. A fusion protein selected from the group consisting of:

(a) an amino acid sequence as set forth in SEQ ID NO: 1;

(b) an amino acid sequence which has an identity of greater than 95% to and has the biological function of the amino acid sequence as set forth in SEQ ID NO: 1; and

(c) an amino acid sequence resulted from substitution, deletion or addition of one or more amino acids of the amino acid sequence as set forth in SEQ ID NO: 1, wherein the amino acid sequence has the biological function of the amino acid sequence as set forth in SEQ ID NO: 1.

2. The fusion protein according to Item 1, which further functionally binds to the chromophore AE of formula (I):

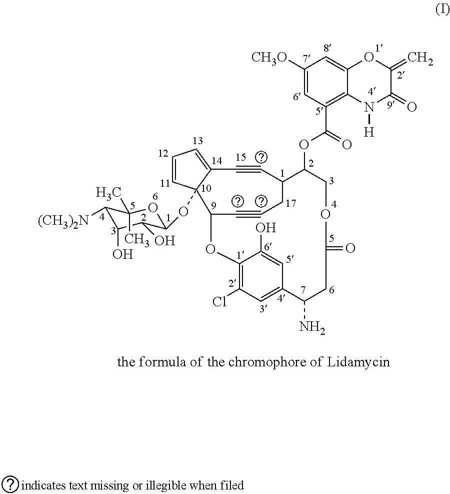

3. A nucleic acid molecule encoding the fusion protein according to Item 1, selected from the group consisting of:

(a) a nucleotide sequence as set forth in SEQ ID NO: 2, which encodes the amino acid sequence as set forth in SEQ ID NO: 1;

(b) the nucleotide sequence encoding the amino acid sequence (b) of Item 1;

(c) the nucleotide sequence encoding the amino acid sequence (c) of Item 1; and

(d) a nucleotide sequence which encodes the same amino acid sequence as SEQ ID NO: 2 but is different from SEQ ID NO: 2 due to codon degeneracy.

4. A vector, which is operably linked to the nucleic acid molecule according to Item 3.

5. The vector according to Item 4, wherein said vector is a plasmid.

6. A host bacterium, comprising the vector according to Item 4.

7. The host bacterium according to Item 6, which has an accession number of CGMCC No. 3125 and is an Escherichia coli named IHPAYZ deposited with China General Microbiological Culture Collection Center (CGMCC) on Jun. 17, 2009.

8. A method for preparing the fusion protein according to Item 2, comprising the following steps:

(a) operably linking the anti-CD20 antibody Fab gene and lidamycin apoprotein LDP gene to the plasmid pCANTAB 5E so as to obtain a recombinant expression plasmid pCANTAB 5E-Fab-LDP,

(b) inducing the expression of the fusion protein Fab-LDP in E. coli. HB2151,

(c) purifying the fusion protein obtained in step (b),

(d) assembling the fusion protein obtained in step (c) with the chromophore of formula (I),

(e) optionally, determining the biological activity of the assembled fusion protein from step (d).

9. A pharmaceutical composition, comprising a pharmaceutically effective amount of the fusion protein according to Item 1 or 2, and optionally, a pharmaceutically acceptable adjuvant.

10. The use of the fusion protein according to Item 1 or 2 in the manufacture of a medicament for tumor-targeted therapy.

11. The use according to Item 10, wherein the medicament is useful for targeting and killing lymphoma cells.

12. The use according to Item 10, wherein the lymphoma is nude mouse lymphoma or human B cell lymphoma.

13. A method for treating a disease, comprising administrating to a subject in need thereof an effective amount of the fusion protein according to Item 1 or 2.

14. The method according to Item 13, wherein said disease is CD20 positive B-cell lymphoma.

15. The method according to Item 13, wherein said disease is non-Hodgkin's lymphoma or diffuse large B-cell lymphoma.

The mode of carrying out the invention is illustrated by the following examples, which are used to explain and illustrate the invention only, rather than limiting the protection scope. Any equivalent variants that can be envisaged by a person skilled in the art according to the common knowledge and the teaching of the prior art fall into the protection scope of the invention.

### EXAMPLES

### Example 1

The construction of the recombinant expression plasmid pCANTAB 5E Fab-LDP

PCR amplification of CH1:

The recombinant plasmid pCANTAB 5E Fcd20 Fab′ contains the VH, VL, and the humanized CL and CH1 gene of the anti-CD20 monoclonal antibody HI47, and in the recombinant plasmid pCANTAB 5E Fcd20 Fab′, only CH1 region contains an apal restriction enzyme cutting site. Therefore, the applicant used the recombinant plasmid pCANTAB 5E Fcd20 Fab′ containing Fab′ gene (Inhibition of human B-cell lymphoma by an anti-CD20 antibody and its chimeric (Fab′)₂ fragment via induction of apoptosis. YinxingLiu, ZhenpingZhu et. al. Cancer Letters 205 (2004)143-153) as the template to obtain CH1. PCR primers were synthesized by Invitrogen, and the corresponding restriction enzyme cutting sites were introduced, respectively.

In particular, the PCR amplification was performed by using pCANTAB 5E Fcd20 Fab′ as the template, P1 as 5′ primer and P2 as 3′ primer. The reaction condition was: an initial denaturation at 94° C. for 5 minutes, followed by 25 cycles of denaturation at 94° C. for 1 minute, annealing at 56° C. for 1 minute, extension at 72° C. for 1 minute. After the last cycle, the extension was performed at 72° C. for 10 minutes. The CH1 gene fragment “A” (about 324 bp) beginning with the apal restriction enzyme cutting site was obtained. The fragment A was run on 1.5% agarose gel electrophoresis and recovered from agarose gel using the type A gel recovery kit from BioDev-Tech. Co.

Anti-CD20Fab′ Upstream Primer P1:

(SEQ ID No: 3)
5′-GCCTCCACCAAGGGCCCATCGGTCTTCCCC-3′

apal restriction enzyme cutting site

Anti-CD20Fab′ Downstream Primer P2:

(SEQ ID No: 4)
5′-CGCGCTGCCACCGCCACCTGTGTGAGTTTTGTCACAAGA-3′

PCR Amplification of LDP:

The PCR amplification was performed by using the recombinant plasmid pET30sngrldp (Accession Number: CGMCC No. 2010) as the template, P3 as 5′ primer, and P4 as 3′ primer. The reaction condition was: an initial denaturation at 94° C. for 5 minutes, followed by 25 cycles of denaturation at 94° C. for 1 minute, annealing at 60° C. for 1 minute, extension at 72° C. for 1 minute. After the last cycle, extension was performed at 72° C. for 10 minutes. The LDP gene fragment B (about 330 bp) was obtained. The fragment B was run on 1.5% agarose gel electrophoresis and recovered from agarose gel using the type A gel recovery kit from BioDev-Tech. Co.

LDP Upstream Primer P3:

(SEQ ID No: 5)
5′-ACAGGTGGCGGTGGCAGCGCGCCCGCCTTCTCCGTC3′

LDP Downstream Primer P4:

(SEQ ID No: 6)
5′-GCGCGCATGCTCAGCCGAAGGTCAGAGCCAC-3′

sphl restriction enzyme cutting site

SOE-PCR Amplification of Fab-LDP:

The amplification was performed by using the purified products of fragment A (CH1) and fragment B (LDP). The reaction condition was: 10 cycles of denaturation at 94° C. for 1 minute, annealing at 60° C. for 1 minute, and extension at 72° C. for 2 minutes, followed by re-extension at 72° C. for 10 minutes. A little CH1-linker-LDP template was produced. After finishing the above reactions, P1 and P4 primers were added to continue the amplification. The reaction condition was: 30 cycles of denaturation at 94° C. for 1 minute, annealing at 60° C. for 1 minute, and extension at 72° C. for 2 minutes, followed by re-extension at 72° C. for 10 minutes. The Fab-LDP gene fragment C (Fragment A+Fragment B, about 669 bp) was obtained.

The product C was run on 1% agarose gel electrophoresis and recovered from agarose gel using the type A gel recovery kit from BioDev-Tech. Co. The recovered Fab-LDP fragment and pCANTAB 5E Fcd20 Fab′ vector were digested by the enzyme apal and sphl. The products were run on 1% agarose gel electrophoresis and recovered from agarose gel using the type A gel recovery kit from BioDev-Tech. Co. The resultant digested products of the vectors were linked to the digested product of the gene of interest at a ratio of 1:6 by T4 ligase (Takara) at a temperature of 16° C. for 16 hours, and then was transformed into the competent E. coli. HB2151. The recombinant clone plasmids were screened and identified by PCR and enzyme digestion, and then were subjected to sequencing. It is shown that for the recombinant expression plasmid of the fusion gene, the results from enzyme digestion and the results from sequencing are completely identical to the results as expected. The sequence is correct and named pCANTAB 5E Fab-LDP. Please see FIG. 1 for the electrophoretogram of the PCR product of the bacteria.

The protein sequence of Fab-LDP is set forth in SEQ ID NO: 1, wherein the amino acid sequence from position 1 to 23 refers to signal peptide; the amino acid sequence from position 24 to 130 refers to variable region of light chain; the amino acid sequence from position 131 to 236 refers to constant region of light chain; the amino acid sequence from position 237 to 358 refers to variable region of heavy chain; the amino acid sequence from position 359 to 466 refers to CH1 region of heavy chain; the amino acid sequence from position 467 to 471 refers to G₄S; and the amino acid sequence from position 472 to 581 refers to lidamycin apoprotein. wherein G₄S refers to a connecting peptide consisting of four glycines and one serine.

The DNA sequence of Fab-LDP is set forth in SEQ ID NO: 2, wherein the sequence from position 1 to 69 refers to the gene sequence of signal peptide; the sequence from position 70 to 390 refers to the gene sequence of light chain variable region; the sequence from position 391 to 708 refers to the gene sequence of light chain constant region; the sequence from position 709 to 1074 refers to the gene sequence of heavy chain variable region; the sequence from position 1075 to 1398 refers to the gene sequence of heavy chain CH1 region; the sequence from position 1399 to 1413 refers to G₄S; the sequence from position 1414 to 1743 refers to the gene sequence of lidamycin apoprotein; the sequence from position 1744 to 1746 refers to terminator codon.

### Example 2

### Expression and Verification of Fab-LDP

2.1 The Expression of Fab-LDP

The single colony of E. Coli. HB2151 containing the plasmid pCANTAB 5E Fab-LDP obtained in Example 1 was seeded in 5 ml 2×YT medium containing ampicillin (Amp) 100 μg/ml, and was incubated overnight under shaking in an constant temperature incubator at 37° C., 200 rpm, and then was transferred into 500 ml 2×YT medium containing Amp 100 μg/ml (1 L 2×YT medium contains 1.6% tryptone, 1.0% yeast extract, 0.5% NaCl, pH 7.4) at 37° C., 200 rpm. After shaking culture for 8 h, the resultant solution was centrifugated by low-temperature high-speed vacuum centrifuge at 6000 rpm, 4′C for 10 min and the bacteria were collected. The bacteria were re-suspended in 1000 ml 2×YT medium containing Amp 100 μg/ml and 1 mM IPTG and were cultured under shaking for 4 h at 30° C., 200 rpm; the bacteria were collected after centrifugation for 10 min at 8,000 rpm, 4° C. and were frozen at −20° C. in refrigerator for further use.

The frozen bacteria were thawed, and 50 ml bacterial periplasmic protein extract (25 mmol/L Tris(Hydroxymethyl)aminomethane, μmol/L ethylenediaminetetraacetic acid (EDTA), 0.1 mmol/L Phenylmethyl Sulfonyl fluorid (PMSF), 20% w/w sucrose, 200 mmol/L NaCl, pH 7.5) was added. The resultant mixture was evenly mixed under shaking, and then was swayed at 4′C for 1 h. The mixture was centrifugated at 12000 rpm, 4′C for 20 min, and the supernatant was taken. After dialyzing the extract with Phosphate Buffered Saline (PBS) for 12 h, the product was purified on fast protein liquid chromatography (FPLC), wherein the affinity chromatographic column was balanced with the binding buffer (0.01 mol/L NaH₂PO₄, 0.01 mol/L Na₂HPO₄, 0.005% NaN₃, pH 7.0), the sample was loaded, the binding buffer was further used to wash the baseline until the baseline was stable, the column was eluting with the eluting buffer (0.1 mmol/L Glycine, pH 3.0), and the eluting buffer was neutralized with the neutralization buffer (1 mol/L Tris-HCl, 0.05% NaN₃, pH 8.2). Then, 12% SDS-PAGE was used to analyze the expression of the foreign protein. The results show that the induced recombinant bacterial strains expressed a lot of foreign proteins, and the expression products of Fab-LDP were mainly present in the soluble periplasm of bacteria (FIG. 2a).

2.2 Identification of Fab-LDP by Western Blotting

The proteins obtained in section 2.1 were electrophoresed. The gel after electrophoresis was subjected to semi-dry-electroporation in the Bio-Rad channel under such a condition that the constant current was 0.7 mA/cm² for 5 hours. After the electroporation finished, poly(vinylidene difluoride)(PVDF) membrane was incubated with the first antibody (i.e., an anti-LDP monoclonal antibody) tenfold-diluted with the blocking buffer (Accession number: CGMCC No. 1849). The horseradish peroxidase(HRP)-labeled Goat Anti-mouse IgG antibody was used as the second antibody. Chromogenic analysis was carried out and the results showed that it was the recombinant fusion protein Fab-LDP carrying LDP at the C-terminal that was expressed in the recombinant bacterial strains (FIG. 2b). The Escherichia coli, which contained the plasmid pCANTAB 5E Fab-LDP and expressed the fusion protein Fab-LDP, was named IHPAYZ and was deposited with China General Microbiological Culture Collection Center on Jun. 17, 2009, wherein the Accession number was CGMCC No. 3125.

### Example 3

### Immune Activity of Fab-LDP

Immunofluorescence binding activity of Fab-LDP in vitro was determined by flow cytometer. 1×10⁶ Raji cells were re-suspended in the solution containing different concentration of FITC(fluorescein isothiocyanate)-labeled anti-CD20 Fab fragment in 100 μL PBS (Inhibition of human B-cell lymphoma by an anti-CD20 antibody and its chimeric (Fab₂) fragment via induction of apoptosis. YinxingLiu, ZhenpingZhu et. al. Cancer Letters 205 (2004) 143-153) or the solution containing Fab-LDP obtained in Example 2 in 100 μL PBS, and was placed at 4° C. for 1 h and centrifugated at 2000 g for 10 minutes. The supernatant was discarded, the leftover was washed with PBS for three times, the positive rate for the anti-CD20Fab fragment or Fab-LDP to bind Raji cell was determined by Fluorescence Activated Cell Sorter (FACS). It was demonstrated that the anti-CD20Fab fragment had substantially the same activity of binding to Raji cells as the Fab-LDP at the same concentration. The Fab-LDP fusion protein retains the ability of binding to the target antigen specifically.

Labeling Fab-LDP with FITC: the Fab-LDP (5 mg/ml) to be cross-linked was dialyzed with the cross-linking reaction liquid at 4° C. for three times until pH was pH 9.0. The formulation of the cross-linking reaction liquid is 7.56 g NaHCO₃, 1.06 g Na₂CO₃, 7.36 g NaCl, wherein water was added to reach a constant volume of 1 L. FITC was dissolved in dimethyl sulfoxide (DMSO) to a concentration of 1 mg/ml. The FITC for cross-linking was immediately formulated before use each time and stored in dark. FITC was slowly added to a solution of Fab-LDP at a ratio of P:F (protein:FITC)=1 mg:150 μg, and at the same time the container was slightly swayed so as to evenly mix the FITC with Fab-LDP. The resulting mixture was placed in the reciprocating shaker to react under shaking at 4° C. in dark for 12 h. 5 mol/L NH₄Cl was added to reach a final concentration of 50 mmol/L. The termination reaction lasted for 2 h at 4° C. The cross-linked product was dialyzed in PBS for 4 times until the dialysate was clear. The identification of the cross-linked product: protein concentration (mg/ml)=[A280−0.31×A495]/1.4; F/P ratio: 3.1×A495/[A280−0.31×A495], the value should be between 2.5 and 6.5.

The antigen-binding activity of Fab-LDP in vivo was measured by in vivo imaging instrument for small animals. 5-6-weekold BALB/c-nu/nu mice, female, weighed 18-20 g (purchased from Beijing Vital River Laboratory. Animal Center, animal permit numbers: SCXK(Jing) 2007-0001) were used. After total body irradiation with Cs ray (dose: 4 Gy/rat), the mice were subcutaneously inoculated with Raji or K565 cells in right armpits, wherein the number of cells was 2×10⁷/0.2 ml for each mouse. When the tumors grew into a size of 60-80 mm³, Fab-LDP, anti-CD20Fab and LDP (16 nmol/rat) were injected via tail vein, respectively. The dynamic distribution of Fab-LDP in the tumor-bearing mice was observed at 1 h, 3 h, and 10 h by small animal in vivo imaging system. The results showed that Fab-LDP or anti-CD20Fab could specifically bind to the tumor tissues expressing CD20 antigen within the first hour; after 3 h, Fab-LDP or anti-CD20Fab began to enrich in the center of the tumor tissues of the tumor-bearing mice, indicating that the monoclonal antibody had entered the center of the tumor; at 10 h, Fab-LDP or anti-CD20Fab continued to enrich in tumor tissues, however, LDP did not specifically bind to the tumor tissues expressing CD20 antigen (FIG. 3b).

### Example 4

### The Preparation of Lidamycin and the Determination of the Relative Content of Its Active Chromophore AE

4.1 The Preparation of Lidamycin (LDM)

0.7 ml salt-free water was added to a dry and cold tube containing the lidamycin-producing bacterium (CGMCC NO. 0135) to form a suspension of bacterium. The suspension was seeded in GAUZE's Medium NO. 1 by virtue of the platinum loop and cultured at 28° C. for 7-10 days. White aerial mycelium grew on the surface. A piece of the aerial mycelium was placed and cultured in the class I seed 100 ml/500 ml conical flask (the fermentation medium comprising 1% starch, 0.5% corn steep liquor, 0.5% peptone, 0.5% glucose, 0.02% MgSO₄, 0.06% KI, 1.5% maize flour, 0.4% CaCO₃, and tap water, pH 7.0, 15 pounds disinfecting) at 28° C. under shaking for 48 h in the rotary shaker. 5% seed was transferred into 1000 ml/5000 ml vertical bottle as the class II seed and was cultured in the same fermentation medium at 28° C. in reciprocating shaker for 18 h. The mixture was loaded in a 200 L fermenter to a volume of 100 L, with an inoculation amount of 2%. 0.03% GPE was added as the antifoaming agent. The mixture was stirred under a tank pressure of 0.04 at 28° C., 400 rpm, airflow 1/1, pH 6.5-7.0, and was fermented for 96 h to get the desired fermentation liquor. 10 L fermentation liquor was centrifugated to get the supernatant. The supernatant was adjusted to pH 4.0 by HCl. 4.5 kg (NH₄)₂SO₄ was added and the resultant solution was stirred at 8° C. for 3 h. The precipitated lidamycin was isolated by centrifugation (4° C., 8000 rpm, 15 min). The precipitate was dissolved in 200 ml cold water, the resultant solution was dialyzed and centrifugated to remove the insolubles. The supernatant was loaded on hydroxyapatite column, and was eluted by 0.001 M phosphate buffer (pH 6.8). The active portion was lyophilized to obtain 1500 mg crude product. The crude product was dissolved in water, after purification by Sephadex G-75 column chromatography, the active portion was lyophilized to obtain 145 mg white power of lidamycin with high anti-tumor activity.

4.2 The Determination of the Relative Content of its Active Chromophore AE

As compared to the protein portion of LDM, the chromophore has a small molecular weight, which only accounts for 7.4% of the molecular weight of lidamycin theoretically. Since AE is the active portion of LDM and the role of apoprotein merely lies in the protection of AE, the activity of the LDM product can be determined by the determination of the relative content of the chromophore AE in the total chromophore of LDM generally.

The percentage of AE in the total amount of chromophore can be determined by analyzing LDM by HPLC. The method is as follows.

The LDM product prepared above was dissolved in the mobile phase of HPLC (the ratio of acetonitrile:water is 23:77). The resultant mixture was isolated on radically-pressured C4 semi-preparative column (Waters) by fast protein liquid chromatography (FPLC), the eluent was acetonitrile:water (23:77), and was collected by automatic collector. The collected components were detected by HPLC C4 analytical column.

The analysis results show that in the LDM prepared by the applicant, the AE component accounts for 90.63% of the total chromophore of LDM. It is determined by analysis that the LDM product meets the quality control standard and is a good LDM product with a high content of AE. The LDM product was lyophilized and stored in refrigerator at −80′C for facilitating the preparation of the energized fusion protein Fab-LDM.

4.3 The Preparation of the Energized Fusion Protein Fab-LDM

10 mg highly active LDM lyophilized product was added to 5 ml cold methanol and was slightly swayed for 5 minutes, and was placed at −20° C. for 1 h with shaking once at the middle of the time. The mixture was centrifugated at 0° C., 12000 rpm for 20 minutes. The supernatant contained the chromophore AE, the precipitate was the protein part of LDM. The extraction was performed twice. The methanol solution containing the chromophore AE was evaporated and concentrated, and was stored at −70° C. Since the chromophore AE was unstable, the experiment was carried out at a low temperature of 4° C. in dark.

The Fab-LDP fusion protein obtained in Example 2 was dissolved in PBS, and the chromophore-methanol solution (5-folded molecular weight) was added (the volume ratio is 50:1). The resultant mixture was mixed under shaking and kept at room temperature for 12 h. Finally, the mixed solution was subjected to PD-10 column chromatography, and the energized fusion protein Fab-LDM was collected after monitoring at A280 nm and A343 nm on Ultraviolet Detector. The Fab-LDM as involved in the following Examples were prepared according to the method.

### Example 5

### The Specific Cytotoxic Effect of Fab-LDM to Tumor Cells

5.1 The Specific Cytotoxic Effect of Fab-LDM to Tumor Cells Cultured In Vitro

The cytotoxic effect was measured by MTT method. Raji or Daudi cells in logarithmic growth phase were counted and seeded on a 96-well plate at 2×10⁴ cells/well and were cultured at 37° C. in an incubator with 5% CO2 for 12 h, and then the Fab-LDM at different concentrations was added, three parallel wells for each concentration. After further culturing for 48 h, the resultant solution was centrifugated at 2000 rpm for 10 minutes and the supernatant was removed. MTT (5 mg/ml) dissolved in 20 μl PBS was added to each well, the cells were cultured at 37° C. for further 4 h, and then centrifugated at 2000 rpm for 10 minutes. The supernatant was slightly discarded and 100 μl dimethyl sulfoxide (DMSO) was added. The 96-well plate was shaken on the shaker at room temperature for 5 minutes. The absorption value was measured on a microplate reader at a wavelength of 546 nm. For each test, three drug-free control wells and three cell-free control wells were set. The survival rate and the half-inhibitory concentration (IC₅₀) were calculated according to the following formula:

survival rate=(Aₜₑₛₜ−A_{blank})/(A_{control}−A_{blank})×100%.

The results showed that the IC₅₀ value of the energized fusion protein Fab-LDM on CD20-positive Raji and Daudi cells were 0.9×10⁻¹⁰ M and 0.8×10⁻¹⁰ M, respectively, and the IC₅₀ value of the energized fusion protein Fab-LDM on CO20-negative K562 cells was 3.0×10⁻¹⁰M, while the IC₅₀ value of the lidamycin on Raji and Daudi cells were 3.1×10⁻¹⁰ M and 2.9×10⁻¹⁰ M, respectively, and the IC₅₀ value of the lidamycin on K562 cells was 2.8×10⁻¹⁰M. This indicated that anti-CD20(Fab)-LDM retained the potent cytotoxic effects of LDM, and selectively killed the tumor cells (see FIG. 4a-d).

5.2 The Specific Cytotoxic Effect of Fab-LDM on Primary Tumor Cells from Patients

The cytotoxic effect was measured by MTT method. Primary tumor cells from patients were counted and seeded on a 96-well plate at 2×10⁴ cells/well and were cultured at 37° C. in an incubator with 5% CO₂ for 12 h, and then the Fab-LDM at different concentrations was added, three parallel wells for each concentration. After further culturing for 48 h, the resultant solution was centrifugated at 2000 rpm for 10 minutes and the supernatant was removed. MTT (5 mg/ml) dissolved in 20 μl PBS was added to each well, and the cells were cultured at 37° C. for further 4 h, and then centrifugated at 2000 rpm for 10 minutes. The supernatant was slightly discarded and 100 μl dimethyl sulfoxide (DMSO) was added. The 96-well plate was shaken on the shaker at room temperature for 5 minutes. The absorption value was measured on a microplate reader at a wavelength of 546 nm. For each test, three drug-free control wells and three cell-free control wells were set. The survival rate and the half-inhibitory concentration (IC₅₀) were calculated according to the following formula:

survival rate=(Aₜₑₛₜ−A_{blank})/(A_{control}−A_{blank})×100%.

The results showed that Fab-LDM retained the cytotoxic effect of LDM and selectively killed primary tumor cells from patients. In particular, it is worth to mention that Fab-LDM also selectively killed the primary cells from the patients resistant to Mabthera (Table 1)

TABLE 1
The inhibitory effect of the fusion protein Fab-LDM
on the primary tumor cells of NHL patients in vitro

| Patient | | diagnostic | CD19⁺ | IC50(μmol/l) | | reisistant to |
|---|---|---|---|---|---|---|
| No. | gender | result | (%) | anti-CD20Fab-LDM | LDM | Mabthera |
| 1 | female | NHL | 30% | 7.2 × 10⁻⁸ | 6.1 × 10⁻⁵ | yes |
| 2 | male | NHL | 27% | 2.0 × 10⁻⁶ | 1.0 × 10⁻⁵ | Undetected |
| 3 | female | DLBCL | 23% | 3.5 × 10⁻⁶ | 2.0 × 10⁻⁵ | Undetected |
| 4 | female | DLBCL | 15% | 9.2 × 10⁻⁸ | 8.0 × 10⁻⁵ | Undetected |

Annotation for the abbreviates: NHL for non-Hodgkin's lymphoma; DLBCL for diffuse large B-cell lymphoma.

### Example 6

### The Therapeutic Effect of Fab-LDM Against CD20⁺ B-Cell Lymphoma Xenografts in Nude Mice

6.1 The Therapeutic Effect of Fab-LDM Against CD20⁺ B-Cell Lymphoma xenografts in nude mice at early stage

The 5 week old BALB/c nude mice weighed 16-18 g in a good state were randomly grouped. After Cs radiation, the nude mice were subcutaneously inoculated with Raji cells in right armpits within 24 h, wherein the number of cells was 2×10⁷ for each mouse and the mice were raised for 7 days. The drugs were administered by i.v. injection (0.2 ml/mouse). After 9 days, the drugs were adminstered again. The therapeutic effect of Fab-LDM on the subcutaneous lymphoma xenografts in nude mice was observed, and the growth curve of the subcutaneous lymphoma xenografts was plotted. For the control group, saline was administered by i.v. injection (0.2 ml/mouse). For the rest groups, different doses of the energized fusion protein Fab-LDM, LDM and 4 nmol/kg anti-CD20Fab were administered by i.v. injection (0.2 ml/mouse). During the test, the long diameter a and short diameter b of the tumor were measured once for every three days, and the weight of nude mice were recorded. The tumor volumes were calculated according to the formula V=0.5 ab², and the tumor-inhibited rates were calculated (FIG. 5a).

The therapeutic results of the energized fusion protein Fab-LDM showed that Fab-LDM at a dose of both 2 nmol/kg and 4 nmol/kg could significantly inhibit or delay the growth of the xenograft lymphoma in nude mice and exhibited stronger ability of inhibiting the growth of tumor as compared to the free lidamycin at a corresponding dose, thereby enhancing the therapeutic effect of lidamycin. The result at the 39^{th} day of the test showed that Fab-LDM at a dose of 4 nmol/kg and 2 nmol/kg had a tumor-inhibition rate of 88% and 73%, respectively, which were higher than which of LDM at the corresponding doses, therein the tumor-inhibition rate of LDM is 69% and 49%. During the therapeutic period of the test, the weight of the animals increased and the general condition was good, indicating that the animals could tolerate the doses used (Table 2).

TABLE 2
The inhibitory effect of Fab-LDM on the growth of CD20⁺ B
lymphoma xenografts at the early stage in nude mice

| | | number of | weight (g) | | |
|---|---|---|---|---|---|
| | | mice at the | at the | tumor | inhibitory |
| | dose | beginning/at | beginning/ | volume | rate |
| Group | (nmol/kg) | the end | at the end | (mm³) | (%) |
| Control | | 5/5 | 16.6/20.4 | 2988.75 | |
| LDM | 4 | 5/5 | 16.58/20.6 | 903.125 | 69^{ΔΔ} |
| | 2 | 5/5 | 16.9/21.2 | 1223.7 | 49^{ΔΔ} |
| Fab-LDM | 2 | 5/5 | 16.25/20.3 | 767.875 | 73^{Δ}* |
| | 4 | 5/5 | 17.3/20.8 | 354.5 | 88^{Δ}* |
| anti- | 4 | 5/5 | 16.08/20.3 | 2880 | |
| CD20Fab | | | | | |

*As compared to LDM, P < 0.05,
^{Δ}as compared to the control, P < 0.05,
^{ΔΔ}as compared to the control, P < 0.01.

6.2 The Therapeutic Effect of Fab-LDM Against CD20⁺ B-Cell Lymphoma xenograftsin nude mice at later stage

The 5 week old BALB/c nude mice weighed 16-18 g in a good state were randomly grouped. After Cs radiation, the nude mice were subcutaneously inoculated with Raji cells in right armpits within 24 h, wherein the number of cells was 2×10⁷ for each mouse. After 25 days, the drugs were administered by i.v. injection. After 9 days, the drug was injected again. The therapeutic effect of Fab-LDM on the subcutaneous lymphoma xenografts in nude mice was observed, and the growth curve of the subcutaneous lymphoma xenografts was plotted (FIG. 5b). For the control group, saline was administered by i.v. injection (0.2 ml/mouse). For the rest groups, different doses of the energized fusion protein Fab-LDM and LDM were administered by i.v. injection, 0.2 ml/mouse. During the test, the long diameter a and short diameter b of the tumor were measured once for every three days, and the weight of nude mice were recorded. The tumor volume was calculated according to the formula V=0.5 ab², and the tumor-inhibition rate was calculated. The therapeutic results of the energized fusion protein Fab-LDM showed that Fab-LDM at a dose of both 2 nmol/kg and 4 nmol/kg could significantly inhibit or delay the growth of lymphoma xenografts in nude mice and exhibited stronger ability of inhibiting the growth of the tumor as compared to the free lidamycin at the corresponding dose, indicating that the good therapeutic effect of lidamycin on lymphoma at later stage. The result at the 47^{th} day of the test showed that Fab-LDM at a dose of 4 nmol/kg and 2 nmol/kg had a tumor-inhibition rate of 79.2% and 67.5%, respectively, which were higher than the tumor-inhibition rates of LDM at the corresponding doses, therein the tumor-inhibition rate of LDM is 65% and 52.1% (Table 3). During the therapeutic period of the test, the weight of the animals increased and the general condition was good, indicating that the animals could tolerate the doses used.

TABLE 3
The inhibitory effect of Fab-LDM on the growth of CD20⁺ B-cell
lymphoma xenografts at the later stage in nude mice

| | | number of | | | |
|---|---|---|---|---|---|
| | | the mice | | | |
| | | at the | weight (g) | | |
| | | beginning/ | at the | tumor | |
| | dose | at the | beginning/ | weight | inhibitory |
| Group | (nmol/kg) | end | at the end | (mm³) x̅ | rate (%) |
| Control | | 5/5 | 16.5/20.4 | 3255.76 | |
| LDM | 2 | 5/5 | 16.8/20.6 | 1559.52 | 52.1^{Δ} |
| | 4 | 5/5 | 16.5/20.4 | 1136.86 | 65^{ΔΔ} |
| Fab-LDM | 2 | 5/5 | 16.2/20.5 | 1057.94 | 67.5^{Δ}* |
| | 4 | 5/5 | 17.3/20.6 | 657 | 79.2^{Δ}* |

*As compared to LDM, P < 0.05,
^{Δ}as compared to the control, P < 0.05,
^{ΔΔ}as compared to the control, P < 0.01.

### Example 7

### The Evaluation of the Cytoxicity of Fab-LDM

7.1 Weight and Survival State

The weight of the mice and the survival state were observed, and side effects of obvious physiological significance were also recorded, such as dandruff, dehydration, somnolence, ataxia, tachypnea and so on. The mice were weighed for each measurement so as to evaluate the cytoxicity of the drug. The results showed that during the whole therapeutic period, no side effect of obvious physiological significance, such as dandruff, dehydration, somnolence, ataxia, tachypnea and so on, was observed in all the experimental groups. After the treatment, the nude mice bearing B-cell lymphoma xenografts showed increased body weight instead of body weight loss in the groups treated with PBS, Fab-LDM 2 nmol/kg. Fab-LDM 4 nmol/kg and LDM 2 nmol/kg, however, the mice showed decreased body weight in the group of LDM 4 nmol/kg (FIG. 6a).

7.2 The Pathological Statistics of the Injured Parts

After the test was finished, the mice were sacrificed. The organs such as heart, liver, spleen, lung, kidney, small intestine were taken, immersed into formalin overnight and stained with hematoxylin-eosin (HE). LEICA QWINV3 system was used to statistically analyze 50 visual fields of 10 sections of multiple organs from each group, and the injured area was calculated from each group. The results showed that no obvious pathological injure was found in the groups treated with PBS, Fab-LDM 2 nmol/kg, Fab-LDM 4 nmol/kg and LDM 2 nmol/kg, however, injures such as slight swelling renal tubules, liver inflammation cell infiltration and decrease in spleen hematopoietic cells were found in the group treated with LDM 4 nmol/kg (Table 4).

TABLE 4
The pathological statistics of the injured parts

| | | Injured area of | Injured area of | Injured area of |
|---|---|---|---|---|
| | dose | spleen* | liver** | kidney** |
| | nmol/ | (%, x̅ ± SD | (%, x̅ ± SD | (%, x̅ ± SD |
| drug | kg | n = 7) | n = 7) | n = 7) |
| PBS | | no change | no change | no change |
| anti-CD20Fab | 4 | no change | no change | no change |
| Fab-LDM | 2 | no change | no change | no change |
| Fab-LDM | 4 | no change | no change | no change |
| LDM | 2 | no change | no change | no change |
| LDM | 4 | 4.2 ± 1.7 | 8.34 ± 2.9 | 2.6 ± 0.34 |

*The statistics of spleen injury = the number of the nucleated cells in 4 nmol/kg LDM-treated group/the number of the nucleated cells in PBS-treated group
**The statistics of the injured area of liver (kidney) = the injured area of liver(kidney)/the total area of liver(kidney)

7.3 The Determination of the Biochemical Index Such as Liver Function, Kidney Function and Blood Routine

After the test was finished, the mice were sacrificed. The blood was sampled after decapitation. A part of the whole blood was subjected to the assay of blood routine after the anticoagulation treatment; serum was extracted from the other part of the whole blood and was subjected to the test for assessment of the liver function and kidney function. The results showed that the liver function and kidney function were almost not affected in the groups treated with Fab-LDM 2 nmol/kg and Fab-LDM 4 nmol/kg, wherein leukocytes were slightly decreased in the group treated with Fab-LDM 4 nmol/kg (FIG. 6b-d).

### The Advantages of the Present Invention

The advantages and positive effects of the present invention are as follows. An energized fusion protein Fab-LDM of anti-CD20 Fab and the anti-tumor antibiotic lidamycin are prepared by applying the method of gene recombination in combination with molecular reconstruction, wherein the resultant fusion protein not only retains the ability of the monoclonal antibody to bind CD20 antigen, but also has a strong tumor-cell specific killing activity, and also exhibits a good anti-tumor effect in assays in vivo. It reaches a new level in terms of the miniaturization of tumor-targeted immune therapeutic drugs and has a potential application prospect.

1. A fusion protein selected from the group consisting of:
(a) an amino acid sequence as set forth in SEQ ID NO: 1;
(b) an amino acid sequence which has an identity of greater than 95% to and has the biological function of the amino acid sequence as set forth in SEQ ID NO: 1; and
(c) an amino acid sequence resulted from substitution, deletion or addition of one or more amino acids of the amino acid sequence as set forth in SEQ ID NO: 1, wherein the amino acid sequence has the biological function of the amino acid sequence as set forth in SEQ ID NO: 1.

2. The fusion protein according to claim 1, which further functionally binds to the chromophore AE of formula (I):

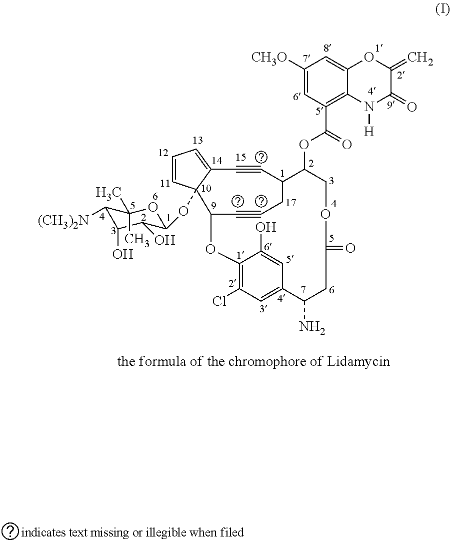

3. A nucleic acid molecule encoding the fusion protein according to claim 1, selected from the group consisting of:
(a) a nucleotide sequence as set forth in SEQ ID NO: 2, which encodes the amino acid sequence as set forth in SEQ ID NO: 1;
(b) the nucleotide sequence encoding the amino acid sequence (b) of claim 1;
(c) the nucleotide sequence encoding the amino acid sequence (c) of claim 1; and
(d) an nucleotide sequence which encodes the same amino acid sequence as SEQ ID NO: 2 but is different from SEQ ID NO: 2 due to codon degeneracy.

4. A vector, which is operably linked to the nucleic acid molecule according to claim 3.

5. The vector according to claim 4, wherein said vector is a plasmid.

6. A host bacterium, comprising the vector according to claim 4.

7. The host bacterium according claim 6, which has an accession number of CGMCC No. 3125 and is an Escherichia coli named IHPAYZ deposited with China General Microbiological Culture Collection Center (CGMCC) on Jun. 17, 2009.

8. A method for preparing the fusion protein according to claim 2, comprising the following steps:
(a) operably linking the anti-CD20 antibody Fab gene and lidamycin apoprotein LDP gene to the plasmid pCANTAB 5E so as to obtain a recombinant expression plasmid pCANTAB 5E-Fab-LDP,
(b) inducing the expression of the fusion protein Fab-LDP in E. coli. HB2151,
(c) purifying the fusion protein obtained in step (b),
(d) assembling the fusion protein obtained in step (c) with the chromophore of formula (I),
(e) optionally, determining the biological activity of the assembled fusion protein from step (d).

9. A pharmaceutical composition, comprising a pharmaceutically effective amount of the fusion protein according to claim 1 or 2, and optionally, a pharmaceutically acceptable adjuvant.

10. The use of the fusion protein according to claim 1 or 2 in the manufacture of a medicament for tumor-targeted therapy.

11. The use according to claim 10, wherein the medicament is useful for targeting and killing lymphoma cells.

12. The use according to claim 11, wherein the lymphoma is nude mouse lymphoma or human B cell lymphoma.

13. A method for treating a disease, comprising administrating to a subject in need thereof an effective amount of the fusion protein according to claim 1 or 2.

14. The method according to claim 13, wherein said disease is CD20 positive B-cell lymphoma.

15. The method according to claim 13, wherein said disease is non-Hodgkin lymphoma or diffuse large B-cell lymphoma.
